(19) United States
(12) Patent Application Publication
(10) Pub. No.: US 2024/0310388 A1
(43) Pub. Date: Sep. 19, 2024

(54) DIAGNOSTIC METHODS FOR HIDRADENITIS SUPPURATIVA

(71) Applicant: AMGEN INC., Thousand Oaks, CA (US)

(72) Inventor: HUIBIN YUE, Thousand Oaks, CA (US)

(73) Assignee: AMGEN INC., Thousand Oaks, CA (US)

(21) Appl. No.: 18/608,576

(22) Filed: Mar. 18, 2024

Related U.S. Application Data
(60) Provisional application No. 63/490,718, filed on Mar. 16, 2023.

(51) Int. Cl. G01N 33/68; A61K 31/445

(52) U.S. Cl. CPC G01N 33/6893; A61K 31/445; G01N 2333/521; G01N 2333/525; G01N 2333/5421; G01N 2800/20; G01N 2800/52; G01N 2800/56

(57) ABSTRACT

The present disclosure provides diagnostic methods, that may be combined with medical treatment, using one or more plasma biomarkers that are useful for identifying a subject with Hidradenitis Suppurativa (HS), measuring disease severity of HS, and measuring clinical response in subjects with HS.

### CROSS-REFERENCES TO RELATED APPLICATIONS

This application claims the benefit of priority under 35 U.S.C § 119(c) to U.S. Provisional Application Ser. No. 63/490,718 filed Mar. 16, 2023, the disclosure of which is incorporated herein by reference in their entirety.

### BACKGROUND OF THE INVENTION

Hidradenitis Suppurativa (HS) is a chronic debilitating skin disease that is graded into Hurley stages I, II, and III based on severity. Hurley H. Dermatologic surgery: principles and practice. 1996:623-645. Hurley stage III is also known as severe Hidradenitis Suppurativa.

Hidradenitis suppurativa (HS), also called acne inversa, is a chronic inflammatory skin disease characterized by inflammatory nodule, abscess, sinus and fistula formation, and scarring of the skin, most commonly in apocrine gland rich areas such as the axilla, inframammary area, inguinal area, perineum, and perianal area. In its moderate and severe forms, HS is debilitating and causes significant discomfort, pain, anxiety and depression, as well as impairment of quality of life.

The exact cause of HS has not been identified, although genetic defects in the gene encoding for gamma-secretase have been described in subjects with HS. Potential target proteins include Notch, E-cadherin, and nicastrin. Notch plays an important role in hair follicle development, and a defect in Notch may lead to formation of epidermal cysts, dysregulation of normal T-cell mediated immune responses, and suppression of Toll-like receptor-4-induced pro-inflammatory macrophage mediated cytokine responses (Radtke et al, 2010; Wang et al, 2010). Smoking and obesity have been associated with HS (Prens and Deckers, 2015) as well as, excessive sweating, androgen dysfunction, or possible genetic causes. Some reports suggest that HS is, at least in part, a neutrophil mediated disease.

Treatment options for subjects with HS includes local and systemic antibiotics, pain medication, and anti-TNF-α agents such as adalimumab. Other drugs such as cyclosporin A, dapsone, and isotretinoin have been used with limited success (Napolitano et al, 2017). Despite the treatment options available, most patients only partially and/or temporarily respond.

Another potential treatment option disclosed in US2018/0280530 and US2018/028425 is the use of a C5a targeting antibody to treat patients suffering from HS.

Further advances in potential treatment options for subjects with severe hidradenitis suppurativa, otherwise known as Hurley Stage III, are disclosed in US2022/125775 that describes certain C5aR inhibitors, such as avacopan, that are especially effective at treating Hurley Stage III subjects in needs of such treatment. Such targeted therapies for Hurley Stage III subjects demonstrate that there is also a need for new and improved methods to identify subjects with Hurley Stage III that can benefit from such a targeted therapy.

There are limited treatment options and methods of diagnosis that are available for subjects with HS, including Hurley Stage III. Therefore, there is an urgent need for new and improved methods to identify HS and Hurley Stage III subjects that can benefit from therapies that are effective for treating these subjects.

### BRIEF SUMMARY

The present disclosure provides various novel diagnostic methods for subjects with HS. In one embodiment, the disclosure relates to methods of testing for Hidradenitis Suppurativa (HS) in a subject comprising measuring one or more plasma biomarkers in a sample collected from the subject, wherein the one or more plasma biomarkers are selected from the group consisting of IL8, IL36β, MDC and TNFα.

In another embodiment, the disclosure relates a method of treating a subject with HS comprising testing for Hidradenitis Suppurativa (HS) in a subject comprising measuring one or more plasma biomarkers in a sample collected from the subject, wherein the one or more plasma biomarkers are selected from the group consisting of IL8, IL36β, MDC and TNFα; and treating the subject by administering a therapeutically effective amount of one or more agents for the treatment of HS.

In another embodiment, the disclosure relates to a method for measuring disease severity of HS in a subject comprising measuring one or more plasma biomarkers in a sample collected from the subject, wherein the one or more plasma biomarkers are selected from the group consisting of APRIL, BAFF, C5b-9, CD25, IL6, IL8 and S100A8/89.

In another embodiment, the disclosure relates to a method of treating a subject with Hurley Stage III comprising measuring disease severity of HS in a subject comprising measuring one or more plasma biomarkers in a sample collected from the subject, wherein the one or more plasma biomarkers are selected from the group consisting of APRIL, BAFF, C5b-9, CD25, IL6, IL8 and S100A8/89; and treating the subject by administering a therapeutically effective amount of one or more agents for the treatment of Hurley Stage III.

In another embodiment, the disclosure relates to a method of measuring HS Clinical Response (HiSCR) rate in a subject with HS that are being treated with an agent that is effective at treating HS comprising measuring one or more plasma biomarkers in a sample collected from the subject, wherein the one or more plasma biomarkers are selected from the group consisting of CD25, Complement Factor B, IL8, NGAL, S100A8/A9 and VEGF.

Other objects, features, and advantages of the present disclosure will be apparent to one of skill in the art from the following detailed description and figures.

### BRIEF DESCRIPTION OF THE DRAWINGS

FIG. 1 shows a schematic of the Phase II study design.

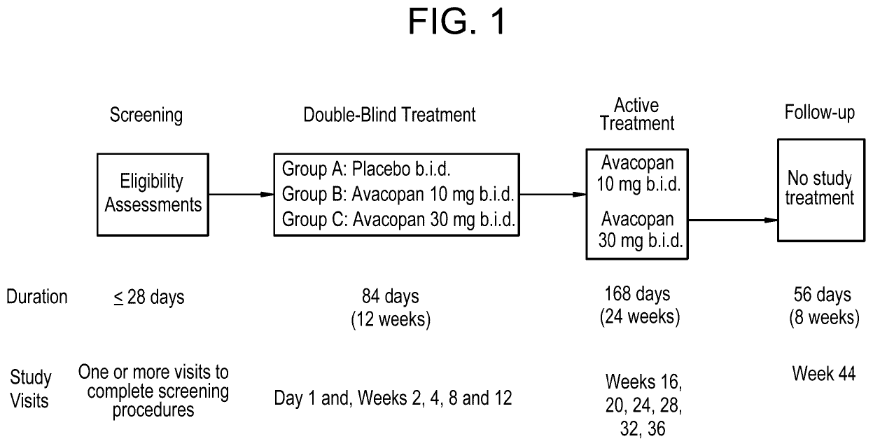

### DETAILED DESCRIPTION OF THE INVENTION

### I. General

This disclosure relates to methods to identify subjects with HS, subjects with Hurley Stage III, methods of treating subjects with HS or Hurley Stage III, companion diagnostics for subjects with HS or Hurley Stage III, and methods of measuring HS clinical response in subjects with Hurley Stage III.

Hurley Staging was developed for the purpose of classifying HS patients by their disease severity, with Hurley Stage III indicating severe disease. Plasma biomarkers for clinical response and disease severity were identified from subjects with HS in the Phase II AURORA Study. A group of biomarkers were identified that could predict the presence of HS in subjects. A second group of biomarkers were identified that could differentiate Hurley Stage III from less severe forms of HS. A third set of biomarkers were identified that could measure HS Clinical Response (HiSCR) rate in a subject with HS.

### II. Definitions and Abbreviations

As used herein, the term “treating” or “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms). Treatment methods provided herein include, in general, administration to a patient an effective amount of one or more compounds provided herein. Suitable patients include those patients suffering from or susceptible to {i.e., prophylactic treatment) a disorder or disease identified herein. Typical patients for treatment as described herein include mammals, particularly primates, especially humans. Other suitable patients include domesticated companion animals such as a dog, cat, horse, and the like, or a livestock animal such as cattle, pig, sheep and the like.

The terms “significant increase,” “significantly increase,” “significant decrease,” and “significantly decrease,” refers to a 5%, 10%, 15%, 20%, or more change in the amount of an analyte between a test sample and a reference sample.

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S.M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, certain compounds of Formula I are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of Formula I can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of Formula I possess asymmetric carbon atoms (optical centers) or double bonds; the racemates, diastereomers, geometric isomers, regioisomers and individual isomers (e.g., separate enantiomers) are all intended to be encompassed within the scope of the present disclosure. Tautomers of the compounds of this disclosure are also intended to be encompassed within the scope of the present disclosure. The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are intended to be encompassed within the scope of the present disclosure.

As used herein, a wavy line, “ [symbol] ”, that intersects a single, double or triple bond in any chemical structure depicted herein, represent the point attachment of the single, double, or triple bond to the remainder of the molecule.

TABLE OF ABBREVIATIONS

| | |
|---|---|
| APRIl | A proliferation-inducing ligand |
| BAFF | B cell activating factor |
| BID | bis in die (twice daily) |
| CD | cluster of differentiation |
| HS | Hidradenitis Suppurativa |
| IL | Interleukin |
| MDC | macrophage-derived chemokine |
| NGAL | neutrophil gelatinase-associated lipocalin |
| SE | standard error |
| TNF | tumor necrosis factor |
| VEGF | vascular endothelial growth factor |
| HiSCR | Hidradenitis Suppurativa Clinical Response |
| CI | confidence interval |
| DF | draining fistula |

### III. Detailed Description of Embodiments

Embodiment 1 of this disclosure relates to a method of testing for Hidradenitis Suppurativa (HS) in a subject comprising measuring one or more plasma biomarkers in a sample collected from the subject, wherein the one or more plasma biomarkers are selected from the group consisting of IL8, IL36β, MDC and TNFα.
Embodiment 2a of this disclosure relates to the method of testing for HS in a subject according to Embodiment 1, wherein the method comprises measuring 3 or more of said plasma biomarkers.
Embodiment 2b of this disclosure relates to the method of testing for HS in a subject according to Embodiment 1, wherein the method comprises measuring IL8, IL36β, MDC and TNFα.
Embodiment 3 of this disclosure relates to the method of testing for HS in a subject according to any one of Embodiments 1-2, wherein the plasma biomarkers are measured directly in plasma with a fluid phase multi-analyte profiling technology.
Embodiment 4 of this disclosure relates to the method of testing for HS in a subject according to any one of Embodiments 1-3, or any sub-embodiments thereof, wherein the one or more plasma biomarkers measured in the subject is compared with a corresponding reference value; and the presence of HS is determined according to the one or more plasma biomarkers whose values significantly increase or decrease from the corresponding reference values.
Embodiment 5 of this disclosure relates to the method of testing for HS in a subject according to Embodiments 4, wherein the one or more plasma biomarkers comprise IL8, and the amount of IL8 is higher in the subject than the corresponding reference value.
Embodiment 6 of this disclosure relates to the method of testing for HS in a subject according to any one of Embodiments 4 or 5, wherein the one or more plasma biomarkers comprise TNFα, and the amount of TNFα is higher in the subject than the corresponding reference value.
Embodiment 7 of this disclosure relates to the method of testing for HS in a subject according to any one of Embodiments 4-6, wherein the one or more plasma biomarkers comprise IL36β, and the amount of IL36β is lower in the subject than the corresponding reference value.
Embodiment 8 of this disclosure relates to the method of testing for HS in a subject according to any one of Embodiments 4-7, wherein the one or more plasma biomarkers comprise MDC, and the amount of MDC is lower in the subject than the corresponding reference value.
Embodiment 9 of this disclosure relates to the method of testing for HS in a subject according to any one of Embodiments 4-8, wherein the corresponding reference value of the one or more plasma biomarkers is the amount of the corresponding plasma biomarker in healthy subjects.
Embodiment 10 of this disclosure relates to a method of treating a subject with HS comprising;
testing for HS in a subject according to the method of any one of Embodiments 1-9, or any sub-embodiments thereof; and
treating the subject by administering a therapeutically effective amount of one or more agents for the treatment of HS.
Embodiment 11 of this disclosure relates to Embodiment 10, wherein the result of the test is indicative for the treatment of HS.
Embodiment 12 of this disclosure relates to Embodiment Error! Reference source not found., wherein TNFα is approximately 1-3 fold higher in the subject with HS; IL36β is about 25% to about 75% lower in the subject with HS; IL8 is about 2-4 fold higher in the subject with HS; and MDC is about 10% to about 50% lower in the subject with HS.
Embodiment 13 of this disclosure relates to any one of Embodiments 10-12, wherein the testing for HS in a subject is a companion diagnostic test before treating the subject, and the result of the test is indicative for the treatment of HS.
Embodiment 13a of this disclosure relates to any one of Embodiments 10-13, wherein the plasma biomarkers are measured by magnetic beads based Luminex assays.
Embodiment 14 of this disclosure relates to any one of Embodiments 10-13, or any sub-embodiments thereof, wherein the one or more agents are selected from the group consisting of a C5aR inhibitor, a C5a inhibitor, adalimumab, antibiotics, a retinoid, spironolactone or finasteride, and metformin.

Non-limiting examples of C5aR inhibitors include avacopan and INF904. Non-limiting examples of C5a inhibitors include vilobelimab and IFX002.
Embodiment 15 of this disclosure relates to Embodiment 14, wherein the agent is a C5aR inhibitor of Formula I:

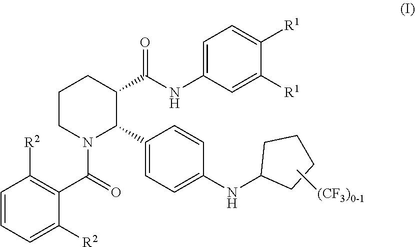

or a pharmaceutically acceptable salt thereof, wherein
each R¹ is independently selected from the group consisting of CH₃, CF₃, CH₂CH₃, Cl, 1-pyrrolidine, —O—CH(CH₃)₂, and CH₂OH;
each R² is independently selected from the group consisting of CH₃ and F; and
the therapeutically effective amount of Formula I that is administered is from 15 mg to about 60 mg twice daily.
Embodiment 15a of this disclosure relates to Embodiment 15, wherein the compound is

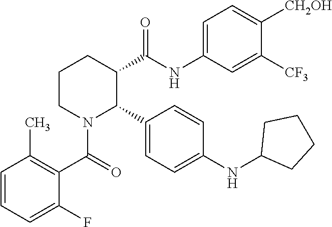 [SMILES: Cc1cccc(F)c1C(=O)N1CCC[C@H](C(=O)Nc2ccc(CO)c(C(F)(F)F)c2)[C@@H]1c1ccc(NC2CCCC2)cc1]

or a pharmaceutically acceptable salt thereof.
Embodiment 16 of this disclosure relates to Embodiment 15, wherein the compound is avacopan, having the formula

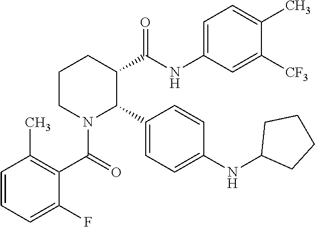 [SMILES: Cc1ccc(NC(=O)[C@H]2CCCN(C(=O)c3c(C)cccc3F)[C@H]2c2ccc(NC3CCCC3)cc2)cc1C(F)(F)F]

or a pharmaceutically acceptable salt thereof.
Embodiment 17 of this disclosure relates to Embodiment 16, wherein the therapeutically effective amount of the compound that is administered is about 30 mg twice daily.
Embodiment 17a of this disclosure relates to Embodiment 16, wherein the subject receives treatment for at least 12 weeks.
Embodiment 18 of this disclosure relates to a method for measuring disease severity of HS in a subject comprising measuring one or more plasma biomarkers in a sample collected from the subject, wherein the one or more plasma biomarkers are selected from the group consisting of APRIL, BAFF, C5b-9, CD25, IL6, IL8 and S100A8/89.
Embodiment 19 of this disclosure relates the method for measuring disease severity of HS in a subject according to Embodiment 18, wherein the method comprises measuring two or more of said plasma biomarkers.
Embodiment 20 of this disclosure relates the method for measuring disease severity of HS in a subject according to Embodiment 19, wherein the method comprises measuring three or more of said plasma biomarkers.
Embodiment 20a of this disclosure relates the method for measuring disease severity of HS in a subject according to Embodiment 20, wherein the method comprises measuring four or more of said plasma biomarkers.
Embodiment 20b of this disclosure relates the method for measuring disease severity of HS in a subject according to Embodiment 20, wherein the method comprises measuring five or more of said plasma biomarkers.
Embodiment 20c of this disclosure relates the method for measuring disease severity of HS in a subject according to Embodiment 20, wherein the method comprises measuring six or more of said plasma biomarkers.
Embodiment 20d of this disclosure relates the method for measuring disease severity of HS in a subject according to Embodiment 20, wherein the method comprises measuring APRIL, BAFF, C5b-9, CD25, IL6, IL8 and S100A8/89.
Embodiment 21 of this disclosure relates the method for measuring disease severity of HS in a subject according to any one of Embodiments 18-20, or any sub-embodiments thereof, wherein the one or more plasma biomarkers measured in the subject is compared with a corresponding reference value; and the disease severity of HS is determined according to the one or more plasma biomarkers whose values significantly increase or decrease from the corresponding reference values.
Embodiment 22 of this disclosure relates the method for measuring disease severity of HS in a subject according to Embodiment 21, wherein the one or more plasma biomarkers comprise APRIL, and a lower amount of APRIL in the subject than the corresponding reference value indicates more severe HS.
Embodiment 23 of this disclosure relates the method for measuring disease severity of HS in a subject according to Embodiment 21 or 22, wherein the one or more plasma biomarkers comprise BAFF, and a lower amount of BAFF in the subject than the corresponding reference value indicates more severe HS.
Embodiment 24 of this disclosure relates the method for measuring disease severity of HS in a subject according to any one of Embodiments 21-23, wherein the one or more plasma biomarkers comprise C5b-9, and a lower amount of C5b-9 in the subject than the corresponding reference value indicates more severe HS.
Embodiment 25 of this disclosure relates the method for measuring disease severity of HS in a subject according to any one of Embodiments 21-24, wherein the one or more plasma biomarkers comprise CD25, and a lower amount of CD25 in the subject than the corresponding reference value indicates more severe HS.
Embodiment 27 of this disclosure relates the method for measuring disease severity of HS in a subject according to any one of Embodiments 21 to 25, wherein the one or more plasma biomarkers comprise IL6, and a higher amount of IL6 in the subject than the corresponding reference value indicates more severe HS.
Embodiment 28 of this disclosure relates the method for measuring disease severity of HS in a subject according to any one of Embodiments 21-26, wherein the one or more plasma biomarkers comprise IL8, and a higher amount of IL8 in the subject than the corresponding reference value indicates more severe HS.
Embodiment 28 of this disclosure relates the method for measuring disease severity of HS in a subject according to any one of Embodiments 21-27, wherein the one or more plasma biomarkers comprise S100A8/89, and a higher amount of S100A8/89 in the subject than the corresponding reference value indicates more severe HS.
Embodiment 29 of this disclosure relates the method for measuring disease severity of HS in a subject according to any one of Embodiments 20-27, or any sub-embodiments thereof, wherein the corresponding reference value of the one or more plasma biomarkers is the amount of the corresponding plasma biomarker in healthy subjects.
Embodiment 30 of this disclosure relates a method of treating a subject with Hurley Stage III comprising:
measuring disease severity of HS according to the method according to any one of Embodiments 18-29, or any sub-embodiments thereof; and
treating the subject by administering a therapeutically effective amount of one or more agents for the treatment of Hurley Stage III.
Embodiment 31 of this disclosure relates the method of treating a subject with Hurley Stage III according to Embodiment 30, wherein the result of the measuring of disease severity of HS is indicative for the treatment of Hurley Stage III.
Embodiment 32 of this disclosure relates the method according to any one of Embodiments 30 or 31, wherein the measuring of disease severity of HS is a companion diagnostic test before treating the subject, and the result of the test is indicative for the treatment of Hurley Stage III.
Embodiment 33 of this disclosure relates the method of treating a subject with Hurley Stage III according to any one of Embodiments 30-32, wherein the plasma biomarkers are measured by a multi-analyte assay.
Embodiment 34 of this disclosure relates the method of treating a subject with Hurley Stage III according to any one of Embodiments 30-33, wherein the plasma biomarker measurement value is compared with a corresponding reference value; and the severity of HS is determined according to Hurley clinical staging system from physician's evaluation.
Embodiment 35 of this disclosure relates the method of treating a subject with Hurley Stage III according to any one of Embodiments 30-34, wherein the agent is selected from the group consisting of a C5aR inhibitor, a C5a inhibitor, adalimumab, antibiotics, a retinoid, spironolactone or finasteride, and metformin.

Non-limiting examples of C5aR inhibitors include avacopan and INF904. Non-limiting examples of C5a inhibitors include vilobelimab and IFX002
Embodiment 36 of this disclosure relates the method of treating a subject with Hurley Stage III according to Embodiment 35, wherein the agent is a C5aR inhibitor of Formula I:

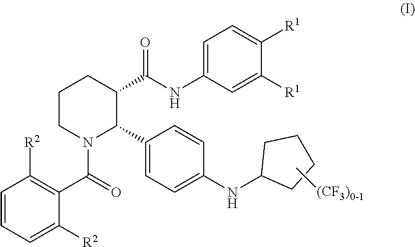

or a pharmaceutically acceptable salt thereof, wherein
each R¹ is independently selected from the group consisting of CH₃, CF₃, CH₂CH₃, Cl, 1-pyrrolidine, —O—CH(CH₃)₂, and CH₂OH;
each R² is independently selected from the group consisting of CH₃ and F; and
the therapeutically effective amount of Formula I that is administered is from 15 mg to about 60 mg twice daily.
Embodiment 36a of this disclosure relates the method of treating a subject with Hurley Stage III according to Embodiment 36, wherein the compound is

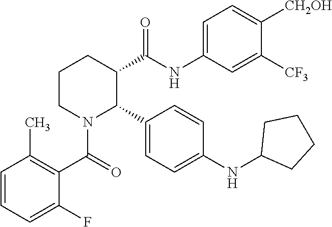 [SMILES: Cc1cccc(F)c1C(=O)N1CCC[C@H](C(=O)Nc2ccc(CO)c(C(F)(F)F)c2)[C@@H]1c1ccc(NC2CCCC2)cc1]

or a pharmaceutically acceptable salt thereof.
Embodiment 37 of this disclosure relates the method of treating a subject with Hurley Stage III according to Embodiment 36, wherein the compound is avacopan, having the formula

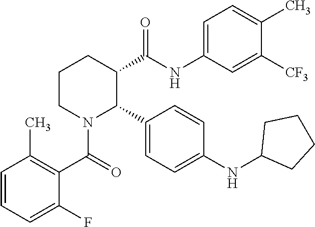 [SMILES: Cc1ccc(NC(=O)[C@H]2CCCN(C(=O)c3c(C)cccc3F)[C@H]2c2ccc(NC3CCCC3)cc2)cc1C(F)(F)F]

or a pharmaceutically acceptable salt thereof.
Embodiment 38 of this disclosure relates the method of treating a subject with Hurley Stage III according to Embodiment 37, wherein the therapeutically effective amount of the compounds that is administered is about 30 mg twice daily.
Embodiment 38a of this disclosure relates the method of treating a subject with Hurley Stage III according to Embodiment 38, wherein the subject receives treatment for at least 12 weeks.
Embodiment 39 of this disclosure relates to a method of measuring HS Clinical Response (HiSCR) rate in a subject with HS that is being treated with an agent that is effective at treating HS comprising measuring one or more plasma biomarkers in a sample collected from the subject, wherein the one or more plasma biomarkers are selected from the group consisting of CD25, Complement Factor B, IL8, NGAL, S100A8/A9 and VEGF.
Embodiment 40 of this disclosure relates to a method of measuring HS Clinical Response (HiSCR) rate in a subject with HS that is being treated with an agent that is effective at treating HS according to Embodiment 39, wherein the method comprises measuring two or more of said plasma biomarkers.
Embodiment 41 of this disclosure relates to a method of measuring HS Clinical Response (HiSCR) rate in a subject with HS that is being treated with an agent that is effective at treating HS according to Embodiment 39, wherein the method comprises measuring three or more of said plasma biomarkers.
Embodiment 41a of this disclosure relates to a method of measuring HS Clinical Response (HiSCR) rate in a subject with HS that is being treated with an agent that is effective at treating HS according to Embodiment 39, wherein the method comprises measuring four or more of said plasma biomarkers.
Embodiment 41b of this disclosure relates to a method of measuring HS Clinical Response (HiSCR) rate in a subject with HS that is being treated with an agent that is effective at treating HS according to Embodiment 39, wherein the method comprises measuring five or more of said plasma biomarkers.
Embodiment 41c of this disclosure relates to a method of measuring HS Clinical Response (HiSCR) rate in a subject with HS that is being treated with an agent that is effective at treating HS according to Embodiment 39, wherein the method comprises measuring CD25, Complement Factor B, IL8, NGAL, S100A8/A9 and VEGF.
Embodiment 42 of this disclosure relates to a method of measuring HS Clinical Response (HiSCR) rate in a subject with HS that is being treated with an agent that is effective at treating HS according to any one of Embodiments 39-41, wherein the plasma biomarkers are measured by a multi-analyte assay.
Embodiment 43 of this disclosure relates to a method of measuring HS Clinical Response (HiSCR) rate in a subject with HS that is being treated with an agent that is effective at treating HS according to any one of Embodiments 39-42, wherein the biomarkers are used to derive a biomarker test that could be given to HS patients to inform treatment with the agent.
Embodiment 44 of this disclosure relates to a method of measuring HS Clinical Response (HiSCR) rate in a subject with HS that is being treated with an agent that is effective at treating HS according to any one of Embodiments 39-43, wherein the agent is selected from the group consisting of a C5aR inhibitor, a C5a inhibitor, adalimumab, antibiotics, a retinoid, spironolactone or finasteride, and metformin.
Embodiment 45 of this disclosure relates to a method of measuring HS Clinical Response (HiSCR) rate in a subject with HS that is being treated with an agent that is effective at treating HS according to Embodiment 44, wherein the agent is a C5aR inhibitor of Formula I:

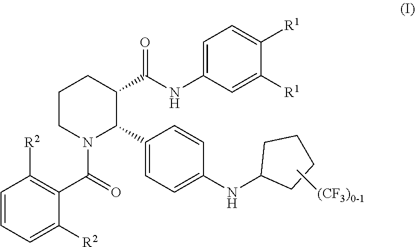

or a pharmaceutically acceptable salt thereof, wherein
each R¹ is independently selected from the group consisting of CH₃, CF₃, CH₂CH₃, Cl, 1-pyrrolidine, —O—CH(CH₃)₂, and CH₂OH;
each R² is independently selected from the group consisting of CH₃ and F; and
the therapeutically effective amount of Formula I that is administered is from 15 mg to about 60 mg twice daily.
Embodiment 45a of this disclosure relates to a method of measuring HS Clinical Response (HiSCR) rate in a subject with HS that is being treated with an agent that is effective at treating HS according to Embodiment 45, wherein the compound is

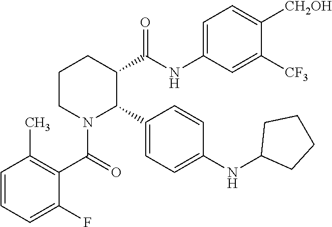 [SMILES: Cc1cccc(F)c1C(=O)N1CCC[C@H](C(=O)Nc2ccc(CO)c(C(F)(F)F)c2)[C@@H]1c1ccc(NC2CCCC2)cc1]

or a pharmaceutically acceptable salt thereof.
Embodiment 46 of this disclosure relates to a method of measuring HS Clinical Response (HiSCR) rate in a subject with HS that is being treated with an agent that is effective at treating HS according to Embodiment 45, wherein the compound is avacopan, having the formula

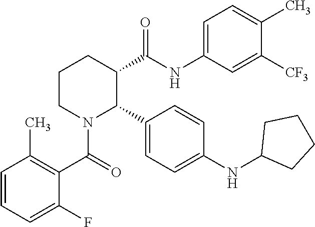 [SMILES: Cc1ccc(NC(=O)[C@H]2CCCN(C(=O)c3c(C)cccc3F)[C@H]2c2ccc(NC3CCCC3)cc2)cc1C(F)(F)F]

or a pharmaceutically acceptable salt thereof.
Embodiment 47 of this disclosure relates to a method of measuring HS Clinical Response (HiSCR) rate in a subject with HS that is being treated with an agent that is effective at treating HS according to Embodiment 46, wherein the therapeutically effective amount of the compound that is administered is about 30 mg twice daily.
Embodiment 47a of this disclosure relates to a method of measuring HS Clinical Response (HiSCR) rate in a subject with HS that is being treated with an agent that is effective at treating HS according to Embodiment 47, wherein the subject receives treatment for at least 12 weeks.
Embodiment 48 of this disclosure relates to a method of treating a subject with Hurley Stage III according to Embodiment 35, wherein the agent is selected from the group consisting of INF904, vilobelimab and IFX002.
Embodiment 49 of this disclosure relates to a method of measuring HS Clinical Response (HiSCR) rate in a subject with HS that is being treated with an agent that is effective at treating HS according to Embodiment 44, wherein the agent is selected from the group consisting of INF904, vilobelimab and IFX002.

### IV. Examples

The following examples are offered to illustrate, but not to limit, this disclosure.

### Example 1—Phase 2 Clinical Study (ClinicalTrials.gov Identifier: NCT03852472)

The study was a randomized, double-blind, placebo-controlled, three group Phase 2 trial in 435 subjects with moderate to severe hidradenitis suppurativa (Hurley stage II or III). Subjects were randomized 1:1:1 to a treatment of 10 mg avacopan twice daily, 30 mg avacopan twice daily or a placebo twice daily for 12 weeks. Following the 12 weeks double-blind treatment period, subjects on placebo were re-randomized 1:1 to receive 10 mg or 30 mg avacopan twice daily for additional 24 weeks. Subjects treated with avacopan continued to receive the same dose (either 10 mg or 30 mg twice daily) for additional 24 weeks.

Subjects were on study treatment for 36 weeks and were followed for 8 weeks for assessment of safety and efficacy. The primary efficacy analysis was at 12 weeks.

The primary efficacy endpoint assessed by the proportion of subjects achieving Hidradenitis Suppurativa Clinical Response (HiSCR) at Week 12.

### Criteria

### Inclusion Criteria:

At least 18 years of age
Clinical diagnosis of HS (Hurley Stage II or III), confirmed by a dermatologist, for at least 6 months prior to Screening
HS lesions are present in at least 2 distinct anatomic areas
Inadequate or loss of response to a systemic course of antibiotics typically of at least 90 days
Must have at least 5 inflammatory nodules or abscesses at screening
Use adequate birth control for subject and partners of child bearing potential
Willing and able to give written Informed Consent

### Exclusion Criteria:

Pregnant or breast-feeding
Any other skin disease that may interfere with the assessment of HS
Rapidly progressive, expanding HS within 30 days prior to screening
More than 20 draining fistulae at screening
Any anti-TNF-α treatment for HS or for other conditions prior to Day 1 visit will be prohibited. Exception: Subjects who were previously treated with an anti-TNF-α drug and discontinued treatment >12 weeks prior to Day 1 visit are allowed for enrollment
Systemic antibiotics are generally excluded
Topical antibiotics use within 14 days prior to Day 1 is excluded
Have started a topical prescription medicine for HS within 14 days prior to screening
A systemic medicine for HS, including biologics and other systemic therapies
Have received within 14 days prior to Day 1 visit or is expected to require oral or transdermal opioid analgesics (except for tramadol) for any reason

The results of NCT03852472 showed that avacopan demonstrated statistically significant dose-dependent improvement in HiSCR (Hidradenitis Suppurativa Clinical Response) vs. placebo in pre-specified Hurley Stage III (severe HS) patients at 12 weeks. There was also a consistent reduction in Hurley Stage III patients in International HS Severity Score (IHS4), as well as reduction in AN (abscesses and inflammatory nodules), draining fistula, and abscess count at week 12. Avacopan was shown to be safe and well tolerated in HS patients.

Surprisingly, it was found that in the Hurley Stage III but not Hurley Stage II subgroups, the percent reduction from Baseline was generally numerically greater for avacopan 30 mg BID group compared to the placebo or avacopan 10 mg BID groups.

The proportion of subjects achieving HiSCR at Week 12 is calculated as those subjects having at least a 50% reduction from baseline in abscess and inflammatory nodule (AN) count, with no increase in abscess count and no increase in draining fistula count number at Week 12 divided by the total number of subjects with non-missing value in each treatment group. p-values are obtained from a Cochran-Mantel-Haenszel (CMH) test stratified by stratification factors Hurley Stage (Stage II vs. III), and anti-TNF drug use (Treatment naïve vs. Previous treatment).

As shown in Table 1, neither avacopan group (10 or 30 mg BID) was superior to the placebo group for HiSCR at Week 12, in the Hurley Stage II subgroup (Table 11). The avacopan 10 mg BID group (21.4%) was statistically inferior to the placebo group (35.3%, p=0.0473).

TABLE 1
HiSCR at Week 12 by Hurley Stage II

| | | | Avacopan | Avacopan |
|---|---|---|---|---|
| | | Placebo | 10 mg BID | 30 mg BID |
| | | N = 85 | N = 84 | N = 87 |
| Hurley | Responder Rate, | 30/80 (35.3) | 18/84 (21.4) | 27/87 (31.0) |
| Stage II | n/N | | | |
| | % Difference | | −13.9 | −4.3 |
| | (avacopan − | | | |
| | placebo) | | | |
| | % Adjusted | | −13.8 | −4.3 |
| | difference | | | |
| | (avacopan − | | | |
| | placebo) | | | |
| | 95% CI | | (−26.8, −0.2) | (−18.1, 9.6) |
| | p-value | | 0.0473 | 0.5469 |

HiSCR in the Hurley Stage III subgroup at Week 12 was significantly higher for the avacopan 30 mg BID group (42.6%) compared to the placebo group (22.2%; p=0.0349). No significant difference was observed between the avacopan 10 mg BID (24.0%) group and the placebo group (22.2%) (Table 2).

TABLE 2
HiSCR at Week 12 by Hurley Stage III (ITT1)

| | | | Avacopan | Avacopan |
|---|---|---|---|---|
| | | Placebo | 10 mg BID | 30 mg BID |
| | | N = 45 | N = 50 | N = 47 |
| Hurley | Responder Rate, | 10/44 (22.2) | 12/50 (24) | 20/47 (42.6) |
| Stage III | n/N | | | |
| | % Difference | | 1.8 | 20.3 |
| | (avacopan − | | | |
| | placebo) | | | |
| | % Adjusted | | 1.8 | 20.8 |
| | difference | | | |
| | (avacopan − | | | |
| | placebo) | | | |
| | 95% CI | | (−15.3, 18.3) | (1.6, 37.9) |
| | p-value | | 0.8397 | 0.8397 |

The clinical effect seen for avacopan 30 mg BID, based on HiSCR at Week 12, in the Hurley Stage III subgroup, is also supported by the observation in Period 1 that the percent reduction from Baseline to Week 12 in AN counts, DF counts, and IHS4 Scores were consistently numerically greater for the avacopan 30 mg BID group as compared to the placebo or avacopan 10 mg BID groups in the Hurley Stage III but not the Hurley Stage II subgroup.

Percent reductions in the Hurley Stage III subgroup for the avacopan 30 mg BID and placebo groups are as below:
Numerically greater percentage reduction in AN count at Week 12 in the avacopan 30 mg BID group (40.8%) compared with the placebo group (23.7%)
Numerically greater percentage reduction in DF count at Week 12 in the avacopan 30 mg BID group (45.2%) compared with the placebo group (24.0%)
Numerically greater percentage reduction in IHS4 Score at Week 12 in the avacopan 30 mg BID group (36.9%) compared with the placebo group (20.7%)

### Example 2—Plasma Biomarkers for Clinical Response and Disease Severity Identified from Hidradenitis Suppurativa Patients in the Phase II AURORA Study

Plasma samples were collected at baseline prior to the start of treatment from 68 enrolled HS patients in phase II AURORA study (NCT03852472) and from 20 independent age and gender matched healthy controls.

TABLE 3
Patients Characteristics in AURORA study

| HS patient samples (n = 68) | |
|---|---|
| Hurley Stage | N |
| II | 49 |
| III | 19 |
| Treatmeants | N |
| Avacopan 30 mg | 24 |
| Avacopan 10 mg | 24 |
| Placebo | 20 |
| HiSCR response rate | % (n/N) |
| Avacopan 30 mg | 54.2 (13/24) |
| Avacopan 10 mg | 29.2 (7/24) |
| Placebo | 25 (5/20) |

The levels of 31 plasma biomarkers listed in Table 4 were assessed including:
Plasma complement factors
Chemokines
Cytokines

TABLE 4
Plasma Biomarkers Assayed

| | |
|---|---|
| 1 | APRIL |
| 2 | BAFF |
| 3 | C3 |
| 4 | C4 |
| 5 | C5 |
| 6 | C5a |
| 7 | C5b-9 |
| 8 | CD163 |
| 9 | CD25/IL2 |
| 10 | CXCL2 |
| 11 | Factor B |
| 12 | FactorI |
| 13 | G-CSF |
| 14 | IL17 |
| 15 | IL1b |
| 16 | IL36b |
| 17 | IL6 |
| 18 | IL8 (HS) |
| 19 | MBL |
| 20 | MDC |
| 21 | MMP-1 |
| 22 | MMP-2 |
| 23 | MMP-8 |
| 24 | MPO |
| 25 | NGAL |
| 26 | Neutrophil Elastase |
| 27 | PDGF-AB/BB |
| 28 | S100A12 |
| 29 | S100A8/A9 |
| 30 | TNFa |
| 31 | VEGF |

The primary objective of this experiment was to Identify a biomarker at baseline to predict or enrich for patients who clinically respond to avacopan (N=68). This experiment included comparing the following groups:
Compared baseline samples from responders and non-responders by treatment group
Compared avacopan 30 mg vs placebo group at week 12

A secondary objective was to identify biomarkers to differentiate Hurley stages II and III, HS and HC by making the following comparisons:
Compare normal donor plasma (N=20) to baseline plasma samples of HS patients (N=68)
Compare baseline plasma samples between the Hurley stage II and Hurley stage III patients

Logistic regression was used to model the clinical response (HiSCR responders vs non-responders) and Hurley stages (II vs III) with natural log transformed baseline biomarker values and treatments (avacopan vs placebo) as predictors. The stepwise procedures were used to identify biomarkers predictors. In stepwise selection, an attempt was made to remove any insignificant biomarkers from the model before adding a significant biomarker to the model. Prior to the first step, the intercept-only model was fit and individual score statistics for the potential variables were evaluated. A significance level of 0.2 is required to allow a variable into the model, and a significance level of 0.25 is required for a variable to stay in the model. Due to a smaller dataset, a selection criteria of p<0.20 was considered here to not eliminate potentially important biomarkers.

As a result of this analysis of these 31 plasma biomarkers, the following are 3 final models were created for the groups of plasma biomarkers that have predictive values based on their statistically significant values (p value<=0.05).

TABLE 5
Plasma Biomarkers that are specific for Identifying HS disease

| Plasma | | | | |
|---|---|---|---|---|
| Biomarkers | Effect Estimate | Std Error | Wald ChiSq test | P values |
| IL8 | 9.131 | 3.963 | 5.309 | 0.021 |
| IL36b | −5.077 | 2.265 | 5.022 | 0.025 |
| MDC | −12.598 | 5.988 | 4.427 | 0.035 |
| TNFa | 13.209 | 5.327 | 6.149 | 0.013 |

The plasma biomarkers IL8, IL36β, MDC and TNFα in Table 6 were measured with multi-analyte technology compatible with plasma (e.g., Luminex xMAP). Fluid phase multi-analyte profiling technology may be based on the principles of a sandwich ELISA, and quantitated with a fluorescent readout converted to pg/ml units using a standard curve. IL8 and TNFα, were measured by Luminex performance human high sensitivity cytokine magnetic panel A (FCSTM09), IL36B was measured by Luminex performance human high sensitivity cytokine magnetic panel B(FCSTM14), and MDC was measured by Luminex human discovery assay (LXSAHM) from R&D systems. The Luminex-based assay is performed according to the manufacture protocol and read by Luminex 100 analyzer.

TABLE 6
Associate with disease severity as
defined by Hurley stage (II vs III)

| Plasma | | | Wald ChiSq | |
|---|---|---|---|---|
| Biomarkers | Effect Estimate | Std Error | test | P values |
| APRIL | −2.85 | 1.33 | 4.61 | 0.03 |
| BAFF | −2.57 | 1.35 | 3.61 | 0.06 |
| C5b_9 | −1.67 | 1.28 | 1.69 | 0.19 |
| CD25_IL2 | −2.29 | 1.37 | 2.78 | 0.10 |
| IL6 | 0.89 | 0.40 | 4.88 | 0.03 |
| IL8 | 1.85 | 0.83 | 4.94 | 0.03 |
| S100A8_A9 | 1.08 | 0.50 | 4.61 | 0.03 |

CD25/IL-2Ra was measured by Quantikine® ELISA assay (DR2A00) and S100A8/A9 was measured by Quantikine® ELISA assay (DS8900) from R&D systems. C5b-9 was measured by QUIDEL Micro Vue C5b-9 EIA (A020). ELISA based assays were performed according to the manufacture protocol and read by flex station 3. April and BAFF, were measured by Luminex Human Discovery Assay (LXSAHM). IL6 was measured by Luminex performance human high sensitivity cytokine magnetic panel B (FCSTM14) and IL8 was measured by Luminex performance human high sensitivity cytokine magnetic panel A (FCSTM09) from R&D systems. The Luminex-based assay was performed according to the manufacture protocol and read by Luminex 100 analyzer.

TABLE 7
Plasma Biomarkers that can predict a clinical benefit
in response to avacopan 30 mg twice daily

| Plasma | | | | |
|---|---|---|---|---|
| Biomarkers | Effect Estimate | Std Error | Wald ChiSq test | P values |
| Placebo | −3.750 | 1.539 | 5.937 | 0.015 |
| CD25_IL2 | 7.486 | 3.232 | 5.365 | 0.021 |
| Factor_B | −4.996 | 2.263 | 4.876 | 0.027 |
| IL8 | −3.940 | 1.557 | 6.406 | 0.011 |
| NGAL | −11.525 | 5.231 | 4.854 | 0.028 |
| S100A8_A9 | 3.363 | 1.513 | 4.940 | 0.026 |
| VEGF | 3.928 | 1.834 | 4.588 | 0.032 |

CD25/IL-2Ra was measured by Quantikine® ELISA assay (DR2A00) and S100A8/A9 was by Quantikine® ELISA assay (DS8900) from R&D systems. ELISA based assays were performed according to the manufacture protocol and read by flex station 3. Complement Factor B were measured by MILLIPLEX® MAP human complement magnetic bead panel 2 (HCMP2MAG-19K) luminex assay from EMD Millipore. IL8 was measured by Luminex performance human high sensitivity cytokine magnetic panel A (FCSTM09) from R&D systems. VEGF was measured by Luminex performance human high sensitivity cytokine magnetic panel A (FCSTM09). NGAL was measured by Luminex human discovery assay (LXSAHM) from R&D systems. The Luminex-based assay was performed according to the manufacture protocol and read by Luminex 100 analyzer.

The interpretation of the Table 5-7 is coming from logistic regression methodology.

The p value<=0.05 is indictive of statistical significance of the biomarker in predicting the outcomes (HiSCR, HS Disease (HS patients vs Health control) and HS Hurley stages (II vs III)).

The “Effect Estimate” (B) measures how much the biomarker can influence the biomarker in predicting the outcomes. For example, in Table 7, increasing the CD25/IL2Ra level by 0.1 unit (in log scale) will increase the odds of achieving HiSCR by 2.11 time.

Increasing the Complement Factor B level by 0.1 unit (in log scale) will get the odds of achieving HiSCR by 0.61 time, (39% odds deduction).

Although the foregoing disclosure has been described in some detail by way of illustration and example for purposes of clarity of understanding, one of skill in the art will appreciate that certain changes and modifications may be practiced within the scope of the appended claims. Where a conflict exists between the instant application and a reference provided herein, the instant application shall dominate.

What is claimed is:

1. A method of testing for Hidradenitis Suppurativa (HS) in a subject comprising measuring one or more plasma biomarkers in a sample collected from the subject, wherein the one or more plasma biomarkers are selected from the group consisting of IL8, IL36β, MDC and TNFα.

2. The method of testing for HS in a subject according to claim 1, wherein the method comprises measuring two or more of said plasma biomarkers.

3. The method according to any one of claims 1-2, wherein the plasma biomarkers are measured directly in plasma with a fluid phase multi-analyte profiling technology.

4. The method according to any one of claims 1-3, wherein the one or more plasma biomarkers measured in the subject is compared with a corresponding reference value; and the presence of HS is determined according to the one or more plasma biomarkers whose values significantly increase or decrease from the corresponding reference values.

5. The method according to claim 4, wherein the one or more plasma biomarkers comprise IL8, and the amount of IL8 is higher in the subject than the corresponding reference value.

6. The method according to claim 4 or 5, wherein the one or more plasma biomarkers comprise TNFα, and the amount of TNFα is higher in the subject than the corresponding reference value.

7. The method according to any one of claims 4-6, wherein the one or more plasma biomarkers comprise IL36β, and the amount of IL36β is lower in the subject than the corresponding reference value.

8. The method according to any one of claims 4-7, wherein the one or more plasma biomarkers comprise MDC, and the amount of MDC is lower in the subject than the corresponding reference value.

9. The method according to any one of claims 4-8, wherein the corresponding reference value of the one or more plasma biomarkers is the amount of the corresponding plasma biomarker in healthy subjects.

10. A method of treating a subject with HS comprising
testing for HS in a subject according to the method of any one of claims 1-9; and
treating the subject by administering a therapeutically effective amount of one or more agents for the treatment of HS.

11. The method according to claim 10, wherein the result of the test is indicative for the treatment of HS.

12. The method according to claim Error! Reference source not found., wherein TNFα is approximately 1-3 fold higher in the subject with HS; IL36β is about 25% to about 75% lower in the subject with HS; IL8 is about 2-4 fold higher in the subject with HS; and MDC is about 10% to about 50% lower in the subject with HS.

13. The method according to any one of claims 10-12, wherein the testing for HS in a subject is a companion diagnostic test before treating the subject, and the result of the test is indicative for the treatment of HS.

14. The method according to any one of claims 10-13, wherein the one or more agents are selected from the group consisting of a C5aR inhibitor, a C5a inhibitor, adalimumab, antibiotics, a retinoid, spironolactone or finasteride, and metformin.

15. The method according to claim 14, wherein the agent is a C5aR inhibitor of Formula I: 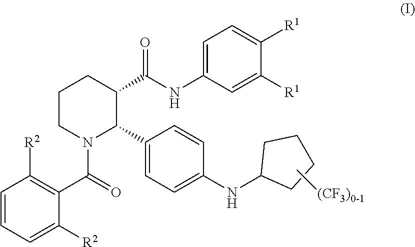
or a pharmaceutically acceptable salt thereof, wherein
each R¹ is independently selected from the group consisting of CH₃, CF₃, CH₂CH₃, Cl, 1-pyrrolidine, —O—CH(CH₃)₂, and CH₂OH;
each R² is independently selected from the group consisting of CH₃ and F; and
the therapeutically effective amount of Formula I that is administered is from 15 mg to about 60 mg twice daily.

16. The method according to claim 14, wherein the compound is avacopan, having the formula 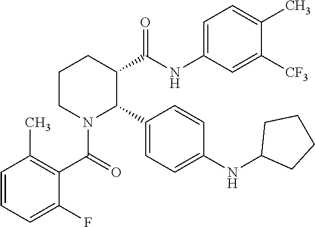 [SMILES: Cc1ccc(NC(=O)[C@H]2CCCN(C(=O)c3c(C)cccc3F)[C@H]2c2ccc(NC3CCCC3)cc2)cc1C(F)(F)F]
or a pharmaceutically acceptable salt thereof.

17. The method according to claim 16, wherein the therapeutically effective amount of the compound that is administered is about 30 mg twice daily.

18. A method for measuring disease severity of HS in a subject comprising measuring one or more plasma biomarkers in a sample collected from the subject, wherein the one or more plasma biomarkers are selected from the group consisting of APRIL, BAFF, C5b-9, CD25, IL6, IL8 and S100A8/89.

19. The method for measuring disease severity of HS in a subject according to claim 18, wherein the method comprises measuring two or more of said plasma biomarkers.

20. The method for measuring disease severity of HS in a subject according to claim 18, wherein the method comprises measuring three or more of said plasma biomarkers.

21. The method for measuring disease severity of HS in a subject according to any one of claims 18-20, wherein the one or more plasma biomarkers measured in the subject is compared with a corresponding reference value; and the disease severity of HS is determined according to the one or more plasma biomarkers whose values significantly increase or decrease from the corresponding reference values.

22. The method for measuring disease severity of HS in a subject according to claim 21, wherein the one or more plasma biomarkers comprise APRIL, and a lower amount of APRIL in the subject than the corresponding reference value indicates more severe HS.

23. The method for measuring disease severity of HS in a subject according to claim 21 or 22, wherein the one or more plasma biomarkers comprise BAFF, and a lower amount of BAFF in the subject than the corresponding reference value indicates more severe HS.

24. The method for measuring disease severity of HS in a subject according to any one of claims 21-23, wherein the one or more plasma biomarkers comprise C5b-9, and a lower amount of C5b-9 in the subject than the corresponding reference value indicates more severe HS.

25. The method for measuring disease severity of HS in a subject according to any one of claims 21-24, wherein the one or more plasma biomarkers comprise CD25, and a lower amount of CD25 in the subject than the corresponding reference value indicates more severe HS.

26. The method for measuring disease severity of HS in a subject according to any one of claims 21 to 25, wherein the one or more plasma biomarkers comprise IL6, and a higher amount of IL6 in the subject than the corresponding reference value indicates more severe HS.

27. The method for measuring disease severity of HS in a subject according to any one of claims 21-26, wherein the one or more plasma biomarkers comprise IL8, and a higher amount of IL8 in the subject than the corresponding reference value indicates more severe HS.

28. The method for measuring disease severity of HS in a subject according to any one of claims 21-27, wherein the one or more plasma biomarkers comprise S100A8/89, and a higher amount of S100A8/89 in the subject than the corresponding reference value indicates more severe HS.

29. The method for measuring disease severity of HS in a subject according to any one of claims 20-27, wherein the corresponding reference value of the one or more plasma biomarkers is the amount of the corresponding plasma biomarker in healthy subjects.

30. A method of treating a subject with Hurley Stage III comprising:
measuring disease severity of HS according to the method according to any one of claims 18-29; and
treating the subject by administering a therapeutically effective amount of one or more agents for the treatment of Hurley Stage III.

31. The method of treating a subject with Hurley Stage III according to claim 30, wherein the result of the measuring of disease severity of HS is indicative for the treatment of Hurley Stage III.

32. The method according to any one of claim 30 or 31, wherein the measuring of disease severity of HS is a companion diagnostic test before treating the subject, and the result of the test is indicative for the treatment of Hurley Stage III.

33. The method of treating a subject with Hurley Stage III according to any one of claims 30-32, wherein the plasma biomarkers are measured by a multi-analyte assay.

34. The method of treating a subject with Hurley Stage III according to any one of claims 30-33, wherein the plasma biomarker measurement value is compared with a corresponding reference value; and the severity of HS is determined according to Hurley clinical staging system from physician's evaluation.

35. The method of treating a subject with Hurley Stage III according to any one of claims 30-34, wherein the agent is selected from the group consisting of a C5aR inhibitor, a C5a inhibitor, adalimumab, antibiotics, a retinoid, spironolactone or finasteride, and metformin.

36. The method of treating a subject with Hurley Stage III according to claim 35, wherein the agent is a C5aR inhibitor of Formula I: 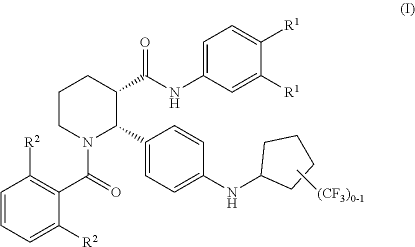
or a pharmaceutically acceptable salt thereof, wherein
each R¹ is independently selected from the group consisting of CH₃, CF₃, CH₂CH₃, Cl, 1-pyrrolidine, —O—CH(CH₃)₂, and CH₂OH;
each R² is independently selected from the group consisting of CH₃ and F; and
the therapeutically effective amount of Formula I that is administered is from 15 mg to about 60 mg twice daily.

37. The method of treating a subject with Hurley Stage III according to claim 36, wherein the compound is avacopan, having the formula 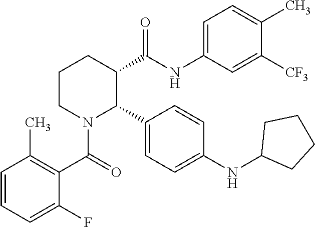 [SMILES: Cc1ccc(NC(=O)[C@H]2CCCN(C(=O)c3c(C)cccc3F)[C@H]2c2ccc(NC3CCCC3)cc2)cc1C(F)(F)F]
or a pharmaceutically acceptable salt thereof.

38. The method of treating a subject with Hurley Stage III according to claim 37, wherein the therapeutically effective amount of the compounds that is administered is about 30 mg twice daily.

39. A method of measuring HS Clinical Response (HiSCR) rate in a subject with HS that is being treated with an agent that is effective at treating HS comprising measuring one or more plasma biomarkers in a sample collected from the subject, wherein the one or more plasma biomarkers are selected from the group consisting of CD25, Complement Factor B, IL8, NGAL, S100A8/A9 and VEGF.

40. The method of measuring HS Clinical Response (HiSCR) rate in a subject with HS that is being treated with an agent that is effective at treating HS according to claim 39, wherein the method comprises measuring two or more of said plasma biomarkers.

41. The method of measuring HS Clinical Response (HiSCR) rate in a subject with HS that is being treated with an agent that is effective at treating HS according to claim 39, wherein the method comprises measuring three or more of said plasma biomarkers.

42. The method of measuring HS Clinical Response (HiSCR) rate in a subject with HS that is being treated with an agent that is effective at treating HS according to any one of claims 39-41, wherein the plasma biomarkers are measured by a multi-analyte assay.

43. The method of measuring HS Clinical Response (HiSCR) rate in a subject with HS that is being treated with an agent that is effective at treating HS according to any one of claims 39-42, wherein the biomarkers are used to derive a biomarker test that could be given to HS patients to inform treatment with the agent.

44. The method of measuring HS Clinical Response (HiSCR) rate in a subject with HS that is being treated with an agent that is effective at treating HS according to any one of claims 39-43, wherein the agent is selected from the group consisting of a C5aR inhibitor, a C5a inhibitor, adalimumab, antibiotics, a retinoid, spironolactone or finasteride, and metformin.

45. The method of measuring HS Clinical Response (HiSCR) rate in a subject with HS that is being treated with an agent that is effective at treating HS according to claim 44, wherein the agent is a C5aR inhibitor of Formula I: 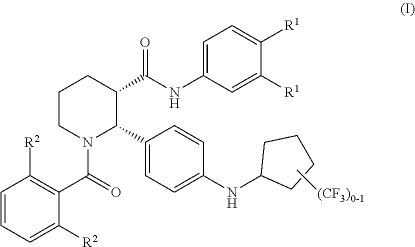
or a pharmaceutically acceptable salt thereof, wherein
each R¹ is independently selected from the group consisting of CH₃, CF₃, CH₂CH₃, Cl, 1-pyrrolidine, —O—CH(CH₃)₂, and CH₂OH;
each R² is independently selected from the group consisting of CH₃ and F; and
the therapeutically effective amount of Formula I that is administered is from 15 mg to about 60 mg twice daily.

46. The method of measuring HS Clinical Response (HiSCR) rate in a subject with HS that is being treated with an agent that is effective at treating HS according to claim 45, wherein the compound is avacopan, having the formula 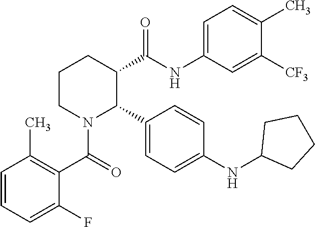 [SMILES: Cc1ccc(NC(=O)[C@H]2CCCN(C(=O)c3c(C)cccc3F)[C@H]2c2ccc(NC3CCCC3)cc2)cc1C(F)(F)F]
or a pharmaceutically acceptable salt thereof.

47. The method of measuring HS Clinical Response (HiSCR) rate in a subject with HS that is being treated with an agent that is effective at treating HS according to claim 46 wherein the therapeutically effective amount of the compound that is administered is about 30 mg twice daily.

48. The method of treating a subject with Hurley Stage III according to claim 35, wherein the agent is selected from the group consisting of INF904, vilobelimab and IFX002.

49. The method of measuring HS Clinical Response (HiSCR) rate in a subject with HS that is being treated with an agent that is effective at treating HS according to claim 44, wherein the agent is selected from the group consisting of INF904, vilobelimab and IFX002.
